(19) United States
(12) Patent Application Publication
(10) Pub. No.: US 2018/0369614 A1
(43) Pub. Date: Dec. 27, 2018

(54) HAIR TREATMENT KIT HAVING A SKIN PROTECTION MEANS

(71) Applicant: Henkel AG & Co. KGaA, Duesseldorf (DE)

(72) Inventors: Hartmut Manneck, Barnitz (DE); Stefan Hoepfner, Hamburg (DE); Matthias Schweinsberg, Hamburg (DE)

(73) Assignee: Henkel AG & Co. KGaA, Duesseldorf (DE)

(21) Appl. No.: 16/064,255

(22) PCT Filed: Dec. 1, 2016
(86) PCT No.: PCT/EP2016/079384

§ 371 Date: Jun. 20, 2018

(30) Foreign Application Priority Data
Dec. 21, 2015 (DE) 10 2015 226 167.0

(51) Int. Cl. A61Q 17/00; A61K 8/73; A61K 8/04; A61Q 5/06; A61K 8/19

(52) U.S. Cl. CPC A61Q 17/00; A61K 8/733; A61K 2800/884; A61Q 5/065; A61K 8/19; A61K 8/042

(57) ABSTRACT

Methods and kits for protecting skin against contact with a hair treatment agent are provided. In an exemplary embodiment, a method for protecting the skin against contact with a hair treatment agent during a hair treatment includes (a) applying a polysaccharide composition (D) onto the hair and (b) applying a gelling composition (C) onto the hair. An exemplary hair treatment kit contains at least one hair treatment agent and at least one agent for protecting the skin against contact with the hair treatment agent, said hair treatment agent being selected from agents for permanent reshaping, straightening or perming, and agents for dyeing keratinous fibers.

### CROSS-REFERENCE TO RELATED APPLICATION

This application is a U.S. National-Stage entry under 35 U.S.C. § 371 based on International Application No. PCT/EP2016/079384, filed Dec. 1, 2016 which was published under PCT Article 21(2) and which claims priority to German Application No. 10 2015 226 167.0, filed Dec. 21, 2015, which are all hereby incorporated in their entirety by reference.

### TECHNICAL FIELD

The present application relates to a hair treatment kit containing at least one hair treatment agent and at least one agent for protecting the skin against contact with the hair treatment agent, said hair treatment agent being selected from agents for permanent reshaping, straightening or perming, and agents for dyeing keratinous fibers.

### BACKGROUND

The cosmetic treatment of skin is an important part of human body care. Nowadays, human hair is treated with hair care cosmetic preparations in numerous ways. Including, for example, cleansing the hair with shampoos, nourishing and regenerating with conditioners and masks, as well as bleaching, dyeing and changing the shape of hair with dyes, toning agents, perming agents and styling preparations. Agents for changing or tinting the color of the hair play a key role in such methods. In blonding agents, an oxidative lightening of the hair is achieved by decomposing the natural hair dyes.

To achieve permanent, intense colors with corresponding fastness properties, so-called oxidative dyes are used. Said dyes usually contain oxidizing dye precursors, so-called developer components and coupler components. The developer components join together or couple with one or more coupler components to form, under the influence of oxidants or atmospheric oxygen, the actual colorants per se. Indeed, the oxidizing dyes are characterized by outstanding, long-lasting color results. To achieve natural-looking colors, however, a mix from a large number of oxidizing dye precursors must typically be used; in many cases, partially-oxidizing dyes are still used to create the tinting effect.

For temporary colors, dyes or tints containing so-called partially-oxidizing agents are normally used as the coloring component. Partially-oxidizing dyes are dye molecules that coat the substrate itself and do not require an oxidative method to create the color. These dyes include Henna, which has been known to color skin and hair since ancient times. Said dyes are usually much more sensitive to shampooing than oxidative dyes, and therefore a highly undesirable shade shift or a visible homogeneous “decoloration” occurs at a much earlier time.

When oxidants are used, the actual dye is usually produced immediately before application by mixing the preparation of the oxidant with the preparation containing the dye precursors. Hydrous hydrogen solutions are predominantly used as oxidants.

Hair dyeing poses several problems—especially when hair is dyed with home applications. For example, it may not be possible to completely remove undesirable staining of skin regions (more particularly the forehead, neck, ear region). Agents for permanent reshaping of the hair can also irritate the skin.

Such skin protection agents are already known from the prior art. These are usually very greasy creams, which are supports to repel the hydrous hair treatment agents. These creams, however, are difficult to wash out.

### BRIEF SUMMARY

Methods and kits for protecting skin against contact with a hair treatment agent are provided. In an exemplary embodiment, a method for protecting the skin against contact with a hair treatment agent during a hair treatment includes (a) applying a polysaccharide composition (D) onto the hair. The polysaccharide composition (D) includes (a1) from about 0.1 to about 5 wt.-%, relative to the total weight of the polysaccharide composition (D), of a polysaccharide adapted to form a gel with calcium ions in a hydrous medium; (a2) from about 50 to about 99.9 wt.-%, relative to the total weight of the polysaccharide composition (D), of water; (a3) optionally at least one alkalizing agent; (a4) optionally at least one polymer, comprising (meth)acrylic acid, (meth)acrylate-, (meth)acrylamide- and/or 2-acrylamido-2-methylpropansulfonic acid monomers; and (a5) optionally from about 0.1 to about 10 wt. %, relative to the total weight of the polysaccharide composition (D), of at least one oil. The method further includes (b) applying a gelling composition (C) onto the hair. The gelling composition includes: (b1) from about 0.1 to about 3 wt. %, relative to the total weight of the gelling composition (C), of at least one salt of a polyvalent metal ion, selected from calcium salt, strontium salt, barium salt and aluminum salt having a water solubility at 20° C. of at least about 500 mg/l; (b2) from about 50 to about 99 wt. %, relative to the total weight of the composition (C), of water; and (b3) optionally at least one cation polymer. The gelling composition (C) has a pH value of from about 4.5 to about 7.5, measured at 20° C. Further, the salts of the gelling composition (C) are not included in the polysaccharide composition (D).

In another embodiment, a kit is provided for hair and skin treatment. The kit includes at least one hair treatment kit compartment containing a hair treatment agent for dyeing and/or for permanently reshaping hair. Further, the kit includes at least one skin protection compartment containing an agent for protecting the skin against contact with the hair treatment agent. The skin protection compartment contains a gelling composition (C) and polysaccharide composition (D). The polysaccharide composition (D) includes (a1) at least one polysaccharide adapted to form a gel with calcium ions in a hydrous medium; (a3) at least one alkalizing agent; and (a4) optionally at least one polymer, comprising (meth)acrylic acid, (meth)acrylate-, (meth)acrylamide- and/or 2-acrylamido-2-methylpropansulfonic acid monomers which are substituted with C₆-C₂₄-alkyl- or C₆-C₂₄-alkenyl groups or ethoxylated C₆-C₂₄-alkyl- or C₆-C₂₄-alkenyl groups. The gelling composition (C) includes (b1) at least one salt of a polyvalent metal ion, selected from calcium salt, strontium salt, barium salt and aluminum salt having a water solubility at 20° C. of at least about 500 mg/l; and (b3) optionally at least one cation polymer.

In another embodiment, a method for protecting skin includes applying to the skin at least one polysaccharide salt selected from calcium alginate, potassium kappa carragheenat, calcium kappa carragheenat, calcium iota carragheenat, calcium pectinate, aluminum alginate, aluminum kappa carragheenat, aluminum iota carragheenat, aluminum pectinate, strontium alginate, strontium kappa carragheenat, strontium iota carragheenat, strontium pectinate, barium alginate, barium kappa carragheenat, barium iota carragheenat, barium pectinate, as well as mixtures of said polysaccharide salts.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure therefore addressed the problem of preparing a skin protection agent for protecting the skin against contact with the hair treatment agents, which are selected from agents for permanent reshaping, more particularly for straightening or perming, and agents for dyeing keratinous fibers, which can easily be washed out and is nonetheless effective.

The subject matter of the present disclosure in a first embodiment is a hair treatment kit, comprising
at least one hair treatment kit compartment containing the hair treatment agent for dyeing and/or for permanently reshaping hair;
at least one skin protection compartment containing the agent for protecting the hair against contact with the hair treatment agent.

The hair treatment kit as contemplated herein contains at least one hair treatment compartment containing the hair treatment agent for dyeing or for permanently reshaping hair and at least one skin protection compartment containing the agent for protecting against contact with the hair treatment agent. In addition, the kit as contemplated herein can comprise other components, including a pre- and/or a post-treatment agent for the treated hair, or more particularly instructions for use, which explains use of the kit as contemplated herein. The kit as contemplated herein is preferably designed and packaged for home use.

The instructions for use preferably comprise an instruction, according to which the user first covers the vulnerable skin or hair regions with the skin protection agent and then applies the hair dye or the hair reshaping agent to the hair. The instructions for use may recommend that after the dyeing or reshaping step, the hair is washed, treated with conditioning agents, etc. The recommendation to rinse out the dye or reshaping agent applied to the hair again after from about 5 to about 40 minutes, preferably approx. 15 to about 30 minutes, is most preferred.

The “kit” as contemplated herein contains at least one hair treatment compartment containing the agent for dyeing or for permanently reshaping hair and at least one skin protection compartment containing the agent for protecting against contact with the hair treatment agent. Said two compartments are connected to one another in the kit according to the present disclosure. To the extent required by the present disclosure, “connected to one another” means any mechanism that explains to a consumer and/or use that the dye and the skin protection agent belong together and are to be applied in combination.

“Connected to one another” therefore means, for example, the collective packaging and the collective sale of the two compartments within a collective outer packaging. Alternatively, the dye and the skin protection agent can be packaged and sold separately, a collective marketing concept recommending collective use to the consumer. This connection can consist in additions, incentive packaging or other consumer benefits upon the purchase of the two products. It can also, however, be achieved by illustrating the other product on the product packaging. Not least, collective advertising for the two products or corresponding instructions for use are also possible.

The instructions for use contained in the kit as contemplated herein can also exist in a non-physical, for example electronic format, for example as a website or as verbal instructions, which can be retrieved by telephone. The addition of data carriers, CD-ROMs or disks, memory sticks, etc., is also possible.

According to the present disclosure, the kit comprises at least one hair treatment compartment. The term “compartment” means here (and also for the skin protection compartment) a receiving possibility, irrespective of the form, material or closure thereof, which includes the possibility of containing substances or substance mixtures. The term “compartment” therefore broadly includes—but is not limited to—the inside of a tube, pouch or bag, a canister, can, tray, bottle, jar, package, carton, box, jacket, viol, shaking bottle or other container. The components of the agent for dyeing hair can (all) be contained in one single compartment. However, it is possible and preferable to offer divide such components among various compartment and to instruct the consumer to mix such components prior to use. Said division is particularly advantageous if the hair-dyeing agent is divided among two components, one containing the basic dye and the other containing the color developer, or if the agent for permanently reshaping hair is divided among two compartments, one containing the keratin-reducing agent (perming or straightening agent) and the other containing the fixing agent.

The at least one hair treatment component contains at least one agent for dyeing or for permanently reshaping hair.

Either partially-oxidizing dyes or oxidizing dyes, which are produced by oxidatively coupling one or more developer components among each other, or with one or more coupler components, are generally used to dye keratinous fibers. Coupler and developer components are also referred to as oxidizing dye precursors.

Primary aromatic amines having another free or substituted hydroxy- or amino group located in a para- or ortho-position, diaminopyridine derivatives, heterocyclic hydrazone, 4-aminopyrazolon derivatives, as well as 2,4,5,6-tetraaminopyrimidine and the derivatives thereof, are typically used as developer components.

Special representatives include p-phenylendiamine, p-toluylendiamine, 2,4,5,6-tetraaminopyrimidine, p-aminophenol, N,N-bis-(2-hydroxyethyl)-p-phenylendiamine, 2-(2,5-diaminophenyl)-ethanol, 2-(2,5-diaminophenoxy)-ethanol, 1-phenyl-3-carboxyamido-4-amino-pyrazol-5-on, 4-amino-3-methylphenol, 2-aminomethyl-4-aminophenol, 2-hydroxymethyl-4-aminophenol, 2-hydroxy-4,5,6-triaminopyrimidine, 2,4-Dihydroxy-5,6-diaminopyrimidine and 2,5,6-triamino-4-hydroxypyrimidine. m-phenylendiamine derivatives, naphthols, resorcin and resorcin derivatives, pyrazolones, m-aminophenols and substituted pyridine derivatives are typically used as coupler components. α-Naphthol, 1,5-, 2,7- and 1,7-dihydroxynaphthalin, 5-amino-2-methylphenol, m-aminophenol, resorcin, resorcinmonomethylether, m-phenylendiamine, 2,4-diaminophenoxyethanol, 2-amino-4-(2-hydroxyethylamino)-anisol (Lehmanns Blue), 1-phenyl-3-methyl-pyrazol-5-on, 2,4-dichlor-3-aminophenol, 1,3-bis-(2,4-diaminophenoxy)-propane, 2-chlorresorcin, 4-chlorresorcin, 2-chlor-6-methyl-3-aminophenol, 2-methylresorcin, 5-methylresorcin, 3-amino-6-methoxy-2-methylamino-pyridine and 3,5-diamino-2,6-dimethoxypyridine are particularly suitable as coupler substances.

With respect to other typical dye components, express reference is made to the “Dermatology” series, published by Ch. Culnan, H. Maibach, Publishing House Marcel Dekker Inc., New York, Basel, 1986, Volume 7, Ch. Zviak, The Science of Hair Care, Chapter 7, pages 248-250 (partially-oxidizing dyes), and Chapter 8, pages 264-267 (oxidizing dyes), as well as the “European Inventory of Cosmetic Raw Materials”, 1996, published by the European Commission, available in disk format from Bundesverband der deutschen Industrie-und Handelsuntemehmen für Arzneimittel, Reformwaren und Körperpflegemittel e.V., Mannheim.

For temporary colors, dyes or tints containing so-called partially-oxidizing agents are normally used as the coloring component. Partially-oxidizing dyes are dye molecules that coat the substrate itself and do not require an oxidative method to create the color. These dyes include Henna, which has been known to color skin and hair since ancient times. Said dyes are usually much more sensitive to shampooing than oxidative dyes, and therefore a highly undesirable shade shift or a visible homogeneous “decoloration” occurs at a much earlier time.

Preferred dyes as contemplated herein contain at least one oxidizing dye precursor of the developer type and, where applicable, at least one oxidizing dye precursor of the coupler type.

The present disclosure is not subject to any restriction at all with respect to the other dye precursors that can be used in the dyes as contemplated herein. The dyes as contemplated herein can be contained in the form of other dye precursors
oxidizing dye precursors of the developer and/or coupler type, and
precursors of natural dyes, such as indol and indolin derivatives,
   as well as mixtures of representatives of said groups.

Preferred agents for dyeing hair preferably contained in the kit as contemplated herein are exemplified in that they contain at least one dye precursor from the groups of aromatic and heteroaromatic diamines, aminophenols, naphthols, polyphenols CH-acid coupler components and the derivatives thereof in quantities of from about 0.01 to about 25 wt. %, preferably from about 0.5 to about 10 wt. %, most preferably from about 1 to about 5 wt. %, relative to the total hair dye in each case.

In addition to the compounds mentioned, the dye contained in the kit as contemplated herein may contain one or more partially-oxidizing dyes for tinting. Partially-oxidizing dyes are typically nitrophenylendiamines, nitroaminophenols, azo dyes, anthrachinones or indophenols. The preferred partially-oxidizing dyes are the compounds known under the international designations and/or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, as well as 1,4-diamino-2-nitrobenzol, 2-amino-4-nitrophenol, 1,4-bis-(β-hydroxyethyl)-amino-2-nitrobenzol, 3-nitro-4-(β-hydroxyethyl)-aminophenol, 2-(2′-hydroxyethyl)amino-4,6-dinitrophenol, 1-(2′-hydroxyethyl)amino-4-methyl-2-nitrobenzol, 1-amino-4-(2′-hydroxyethyl)-amino-5-chlor-2-nitrobenzol, 4-amino-3-nitrophenol, 1-(2′-ureidoethyl)amino-4-nitrobenzol, 4-amino-2-nitrodiphenylamine-2′-carboxylic acid, 6-nitro-1,2,3,4-tetrahydrochinoxaline, 2-hydroxy-1,4-naphthochinon, pikramic acid and the salts thereof, 2-amino-6-chloro-4-nitrophenol, 4-ethylamino-3-nitrobenzoic acid and 2-chloro-6-ethylamino-1-hydroxy-4-nitrobenzol.

The agent for dyeing hair contained in the kit as contemplated herein may also contain all active ingredients, additives and excipients known for such preparations. In many cases, the dyes contain at least one surfactant, wherein both anionic and also zwitterionic, ampholytic, non-ionic and cationic surfactants are suitable in principle. In many cases, however, it has proven advantageous for the surfactants to be selected from anionic, zwitterionic or non-ionic surfactants.

The agents for dyeing hair contained in the kit as contemplated herein preferably contain the active substances in a suitable hydrous, alcoholic or hydrous-alcoholic carrier. Carriers such as creams, emulsions, gels or surfactant-containing, foaming solutions, such as shampoos, foaming aerosols, foam formulations or other preparations suitable for application on the hair, are used for dyeing the hair. However, it is also conceivable for the dye precursors to be integrated into a powdery or a tablet-like formulation.

To the extent required by this present disclosure, anhydrous-alcoholic solutions are anhydrous solutions containing from about 0.1 to about 70 wt. % C₁-C₄ alcohol, more particularly ethanol and/or isopropanol. The agents as contemplated herein can also contain other organic solvents, such as methoxybutanol, benzyl alcohol, ethyldiglycol or 1,2-propylene glycol. All water-soluble organic solvents are preferred.

The pH value of the agent for dyeing hair contained in the kit as contemplated herein can be set within a broad range by employing suitable constituents, such as acidifying agents or alkalizing agents. Preferred agents as contemplated herein are exemplified in that the pH value of the agent is from about 6 to about 11, preferably from about 7.5 to about 10 and most preferably from about 8 to about 9.

In the presence of oxidizing dye precursors, an oxidative dyeing of fibers can in principle take place with atmospheric oxygen. However, a chemical oxidizing agent other than atmospheric oxygen is preferably used. This is the case particularly if, in addition to the dyeing, a lightening effect on human hair is desired. This lightening effect can be desirable, irrespective of the dyeing method. Accordingly, the presence of oxidizing dye precursors is not absolutely essential to the use of oxidizing agents in the agents as contemplated herein. Persulfates, chlorites and, more particularly, hydrogen peroxide or the adducts thereof on urea, melamine and sodium borate can be considered as oxidants.

When oxidants are used, the actual dye is expediently produced immediately before application by mixing the preparation of the oxidant with the preparation containing the compounds of Formula I and, where applicable, dye precursors. The ready-to-use hair dye preparation produced as a result should preferably have a pH value within the range of from about 6 to about 12. The application of the hair dye in a weakly alkali environmental is most preferred. The application temperature can be within a range of between from about 15 and about 40° C. After an application time of from about 5 to about 45 minutes, the hair dye is removed from the hair being colored by rinsing out. The after-washing with a shampoo is not required if a strongly surfactant-containing carrier, e.g. a color shampoo, has been used.

Particularly in the case of hair that is difficult to color, an agent for dyeing hair contained in the kit as contemplated herein can if necessary be applied to the hair with additional dye precursors, but also without prior mixing with the oxidizing component. The oxidizing component is applied after an application time of from about 20 to about 30 minutes—after any necessary intermediate rinsing. After an application time of from about 10 to about 20 minutes, the hair is then rinsed and after-shampooed if desired. In the case of this embodiment, according to a first variant—wherein the prior application of the hair precursors is supposed to improve penetration into the hair—the corresponding agent is set to a pH of approx. 4 to about 7. According to a second variant, oxidation by air is the primary objective, the applied agent preferably having a pH value from about 7 to about 10. During the subsequent accelerated post-oxidation method, the use of acidic peroxide sulfate solutions as the oxidant may be preferred.

The kit as contemplated herein also comprises at least one skin protection compartment containing at least one skin protection agent. Said skin protection agent is applied to the potentially vulnerable skin regions before the dye or reshaping agent is used.

The agent for protecting against contact with the hair treatment agent prevents the dye or reshaping agent subsequently applied from penetrating the skin and/or hair regions that were treated with the skin protection agent.

The agent as contemplated herein for protecting the hair against contact with the hair treatment agent comprises a two-part kit from the following components, (D) and (C), which are physically separate from one another:

I. A polysaccharide composition (D), containing
d)i. at least one polysaccharide, which can form a gel with calcium ions in an aqueous medium and which is preferably selected from alginic acid, kappa carragheenan, iota carragheenan and pectin, and/or at least one salt of the aforementioned polysaccharides, selected from the alkali metal-, ammonium-, mono-, di- and trialkyl ammonium-, mono-, di-and trialkanolammonium- and magnesium salts, wherein in the case of salts of the kappa carragheenan, the alkali metal salt thereof is selected only from lithium- and sodium salts, mixtures of alginic acids and sodium alginate being most preferred,
d)ii. at least one alkalizing agent, preferably selected from powdery sodium silicates, most particularly powdery sodium meta silicates,
d)iii. optionally, at least one polymer, containing (meth)acrylic acid-, (meth)acrylate-, (meth)acrylamide- and/or 2-acrylamido-2-methylpropansulfonic acid monomers, preferably having no monomers, which are substituted with possibly ethoxylated, C₆-C₂₄-alkyl- or C₆-C₂₄-alkenyl groups;
   II. a gelling composition (C′), containing
c)i. at least one salt of a polyvalent metal ion selected from calcium-, strontium-, barium- and aluminum salts having a water solubility at about 20° C. of at least about 500 mg/l, and also if the polysaccharide, which forms a gel with calcium ions, comprises kappa carragheenan or a salt thereof, at least one salt selected from potassium-, calcium-, strontium-, barium- and aluminum salts having a water solubility at about 20° C. of at least about 500 mg/l;
c)ii. optionally at least one cation polymer, which is preferably selected from cationized guar ethers.

From the at least one selected salt of a polyvalent metal ion, selected from calcium-, strontium-, barium- and aluminum salts and under certain preconditions selected from potassium salts, in each case having a water solubility at about 20° C. of at least about 500 mg/l, and from the at least one selected polysaccharide, a gelled film, which acts as a barrier and prevents or at least reduces contact between the skin and the dyeing or reshaping hair treatment agent, forms on the treated skin.

A further subject matter of the present application is a method for protecting the skin against contact with a hair treatment agent during the hair treatment method, said method being selected from permanent reshaping, more particularly for straightening or perming, and the dyeing of keratinous fibers, exemplified in that the following method steps are applied in an arbitrary sequence before the hair treatment agent is applied to the hair:
(i) Application, more particularly spraying, of a polysaccharide composition (D) onto the hair, containing
iii.1 at least one polysaccharide, which can form a gel with calcium ions in a hydrous medium and which is preferably selected from alginic acid, kappa carragheenan, iota carragheenan and pectin, and/or at least one salt of the aforementioned polysaccharides, selected from the alkali metal-, ammonium-, mono-, di- and trialkyl ammonium-, mono-, di-and trialkanol ammonium- and magnesium salts, preferably in a total quantity of from about 0.1 to about 5 wt.-%, more preferably from about 0.5 to about 2 wt.-%, most preferably from about 0.5 to about 2.5 wt. %, in each case relative to the weight of the composition (D), wherein the case of salts of the kappa carragheenan, the salt thereof is selected only from lithium- and sodium salts,
iii.2 from about 50 to about 99.9, preferably from about 60 to about 95, most preferably from about 80 to about 90 wt. % water, in each case relative to the weight of the composition (D),
iii.3 optionally at least one alkalizing agent,
iii.4 optionally at least one polymer, containing (meth)acrylic acid, (meth)acrylate-, (meth)acrylamide- and/or 2-acrylamido-2-methylpropansulfonic acid monomers, preferably in a total quantity of from about 0.05 to about 3 wt. %, more preferably from about 0.2 to about 0.5 wt. %, relative to the weight of the composition (D),
iii.5. optionally at least one oil in a total quantity from about 0.1 to about 10 wt. %, preferably from about 0.3 to about 5 wt. %, more preferably from about 1 to about 3 wt. %, in each case relative to the weight of the polysaccharide composition (D), the polysaccharide composition (D) optionally being produced by mixing a solid, preferably powdery polysaccharide composition (D′), containing iii.1 and optionally iii.3 and/or optionally iii.4, with the component iii.2 and optionally with the component iii.5 in situ or from about 0.01 to about 24 hours before the hair treatment method is applied,
(ii) Application, more particularly spraying, of a gelling composition (C) onto the hair, containing
d)i. from about 0.1 to about 3 wt. %, preferably from about 0.2 to about 2 wt. %, more preferably from about 0.4 to about 1 wt. %, relative to the weight of the composition (C), at least one salt of a polyvalent metal ion, selected from calcium-, strontium-, barium- and aluminum salts having a water solubility at about 20° C. of at least about 500 mg/l, and also if the polysaccharide, which forms a gel with calcium ions, comprises kappa carragheenan or a salt thereof, the gelling composition (C) containing from about 0.1 to about 3 wt. %, preferably from about 0.2 to about 2 wt. %, more preferably from about 0.4 to about 1 wt. %, relative to the weight of the composition (C), at least one salt, selected from potassium, calcium-, strontium-, barium- and aluminums salts having a water solubility at about 20° C. of at least about 500 mg/l,
d)ii. from about 50 to about 99 wt. %, relative to the weight of the composition (C), water,
d)iii. optionally at least one cation polymer, which is preferably selected from cationized guar ethers.
d)iv. wherein the gelling composition (C) has a pH value in the range from about 4.5 to about 7.5, preferably in the range of from about 5 to about 7, most preferably from about 5.5 to about 6.5, measured at about 20° C.; the gelling composition (C) optionally being produced in-situ or from about 0.01 to about 24 hours hours before the hair treatment method is applied by mixing a solid, preferably powdery gelling composition (C′), containing the at least one salt mentioned under d)i in powder form and optionally at least one cation polymer, which is preferably selected from cationized guar ethers, in powder form, with water d)
   wherein (D) and (C) can be applied to the skin in an arbitrary sequence, preferably (D) first and then the gelling composition (C),
   wherein the salts of the gelling composition (C) are not contained in the polysaccharide composition (D).

With this method as contemplated herein, the potentially vulnerable skin regions and/or parts of the head hair are at least partially covered with compositions (D) and (C) before the hair treatment agent is applied to the hair, in order to achieve a protection prior to dyeing and/or the effect of the keratin-reducing reshaping agent.

The skin treatment agent can be formulated as a cream, lotion, gel, liquid, etc. Readily-distributable presentation forms, sprayable formulations having proven particularly suitable, are preferred with respect to applicability. Among such formulations, more particularly low-viscosity preparations, preferably as part of a spraying or foaming method, can be applied to the hair to be protected. Spray- or foam preparations, which can be applied to the hair by employing propellants or by a pump mechanism, are preferred here. Preferred hair treatment kits as contemplated herein are exemplified in that the skin protection compartment is embodied as a spray applicator.

The propellant or propellant mixture is preferably contained in the compositions in a quantity of at least about 1 wt. %. A content of from about 1 to about 10 wt. % and more particularly from about 3 to about 4 wt. % is most preferred. The propellant or propellant mixture preferably contains at least one component which, at normal pressure, has a boiling point in the range of from about 0 to about 40° C., preferably from about 15 to about 35° C. and most preferably from about 20 to about 30° C. The propellant preferably contains a C₅-alkane. Among these—n-pentane, 2-methyl butane (=isopentane) and neopentane—the 2-methyl butane having a boiling point of about 29° C. is most suitable. A mixture of a C₄-alkane and a C₅-alkane is preferably used as the propellant, wherein as contemplated herein, from about 1:3 to about 3:1 mixtures (mixture ratio) from isopentane/isobutane are particularly suitable.

The agents used in the kit as contemplated herein are preferably bottled in commercially available bottling stations, together with the propellant or propellant mixture in the liquefied by the pressure, in dispenser systems, said systems having the pressure resistance required for the propellant to exist in the propellant in liquefied form A container having a valve from a rigid material, which is equipped with a device that uses the contraction force of an extended rubber hose and/or of an extended product container to eject the composition (i.e. agent and propellant) stored therein, is suitable as a dispenser for the composition. The containers offered by 3D Dispenser-Distributions GmbH under the designation FlexPack® also use the recoil force of an extended rubber element to eject the composition. Preferred suitable containers as contemplated herein are those containing a folded, essentially gas-impermeable flexible inner pouch from a chemically-inert plastic (e.g. PET), said pouch being surrounded by an elastic, thick-walled rubber hose.

Most preferred suitable kits as contemplated herein are exemplified in that the skin protection preparation(s) are formulated as aerosol products.

The low-viscosity characteristic of the skin protection agent is most preferred. According to the present disclosure, hair treatment kits with the agent for protection against staining having a viscosity (Brookfield viscometer LVT-II, Spindle 31, about 30 rpm, about 20° C.) from about 0.01 to about 100,000 mPas, preferably from about 0.1 to about 10,000 mPas and most preferably from about 1 to about 1000 mPas, are preferred.

Both the dye contained in the hair treatment compartment(s), and also the skin protection agent contained in the skin protection department, may contain other typical cosmetic ingredients.

Preferred hair treatment kits as contemplated herein additionally contain instructions for use, which instruct the consumer to avoid undesirable staining of skin and/hair using the agent for protection against staining.

As a further component, the aforementioned kit as contemplated herein comprises instructions for use, which give the consumer application instructions on the agents as contemplated herein. To the extent required by the present application, “instructions for use” are not restricted to just written statements in any language. They can also be illustrations, such as pictograms for example.

The instructions for use can be permanently attached to the packaging or affixed thereon. In an embodiment of the present disclosure, for example, the instructions for use can be affixed to the outside of the packaging. In preferred embodiments of the present disclosure, the instructions for use can also be affixed to the inside of the packaging. Such embodiments are recommended in particular for kits as contemplated herein, which have outer packaging.

To the extent required by the present disclosure, instructions for use are “affixed” to packaging when they are firmly adhered to the packaging. In the simplest case, the instructions for use can be printed onto the outside and/or onto the inside of the packaging directly. However, it is possible and preferable for separately pre-prepared instructions for use, which can be scented, to be firmly adhered to the packaging. Possibilities include affixing labels, shrinking-on foils, the mechanical fastening of labels, paper, cartons, cards, etc. by employing click-on, snap-on or clip-on connections.

It is of course also possible to embody the instructions for use as a separate insert, which is placed inside the packaging. Such embodiments are recommended in turn for kits as contemplated herein, which have outer packaging. The separate instructions for use can be prepared on paper, carton, plastics, etc. in the form of sheets, pages, cards, small cards, flyers, fanfold paper, etc. The advantage of the separate instructions for use is that the consumer can fasten them to an exposed place and read them repeatedly during use of the agent. Such separate instructions for use can also be removed from the packaging and read repeatedly from various places, without the entire kit having to be taken along.

Kits as contemplated herein, wherein the instructions for use are embodied as a separate insert, are a further preferred embodiment of the present disclosure.

Preferably, the instructions for use instruct the consumer to apply the agent for protecting against staining to the skin regions vulnerable to staining and/or to hair regions to be protected from decoloration, before the actual dyeing method is applied. The instructions for use can also instruct the consumer to wash off the dye again after an application time following the dyeing method. A further agent for rinsing out (shampoo, conditioning shampoo, etc.), to which reference is made in the instructions for use, can be added to the kit.

Regardless of which method variant is being applied, methods involving the hair being rinsed out after the dyeing or reshaping method are preferred as contemplated herein.

Preferred methods as contemplated herein are exemplified in that the hair dye contains at least one partially-oxidizing dye and/or at least one oxidizing dye precursor. The corresponding compounds are described in detail above. Here too, the corresponding compounds are described in detail above.

The statements made above for the kit as contemplated herein apply mutatis mutandis in all cases.

According to the present disclosure, the expressions “keratinous fibers” and “keratin fibers” are preferably human hair, but can also be fur, wool and feathers.

To the extent required by the present disclosure, “subsequent” is preferably a period from about 1 to about 600 seconds.

### Gel-Forming Salt

An essential component of preferred skin protection agent(s) as contemplated herein is at least one salt of a polyvalent metal ion, selected from calcium-, strontium-, barium- and aluminum salts having a water solubility at about 20° C. of at least about 500 mg/l and, under certain conditions, also selected from potassium salts having a water solubility at about 20° C. of at least about 500 mg/l.

Said polysaccharide, which can form a gel with calcium ions, is preferably selected from alginic acid, kappa carragheenan, iota carragheenan and pectin, and/or at least one salt of the aforementioned polysaccharides, selected in turn from the alkali metal-, ammonium-, mono-, di- and trialkyl ammonium-, mono-, di- and trialkanolammonium- and magnesium salts, wherein in the case of salts of the kappa carragheenan, the alkali metal salt thereof is selected only from lithium- and sodium salts.

At about 20° C., the at least one gel-forming salt has a water solubility of at least about 500 mg/l, preferably of at least about 40 g/l, more preferably of at least about 200 g/l, and most preferably of at least about 350 g/l. The preferred calcium chloride as contemplated herein has a water solubility of about 740 g/l at about 20° C. Other most preferred gel-forming salts as contemplated herein are selected from calcium acetate (water solubility about 374 g/l), calcium lactate, calcium gluconate, calcium gluconate lactate. Aluminum chloride, aluminum hydroxychloride, aluminum sulfate, aluminum lactate, aluminum acetate, aluminum gluconate are likewise preferred. Potassium chloride, potassium acetate, potassium lactate, potassium gluconate and potassium sulfate are preferred for the gelling of kappa carragheenan, potassium, chloride, potassium lactate, and potassium gluconate being most preferred. Strontium chloride and barium chloride are also suitable. Preferably, mixtures of the aforementioned salts can also be contained.

### Polysaccharide, Selected from Alginic Acid, Kappa Carragheenan, Iota Carragheenan and Pectin, and/or at Least One Salt of said Polysaccharides

A further essential component of skin protection method(s) as contemplated herein is at least one polysaccharide, which forms a gel with calcium ions in hydrous medium. Polysaccharides preferred as contemplated herein, which can form a gel with calcium ions in a hydrous medium, are preferably selected from alginic acid, kappa carragheenan, iota carragheenan and pectin, as well as at least one salt of the aforementioned polysaccharides, selected from the alkali metal-, ammonium-, mono-, di- and trialkyl ammonium-, mono-, di-and trialkanolammonium- and magnesium salts, wherein in the case of salts of the kappa carragheenan, the alkali metal salt thereof is selected only from lithium- and sodium salts. Hydrous solutions of said polysaccharides and polysaccharide salts demonstrate, depending on concentration, only a slightly increased viscosity, but do not yet form a solid gel.

Alginic acid, sodium alginate, ammonium alginate, magnesium alginate, monoethanolammonium alginate, kappa carragheenan, the lithium salt of kappa carragheenan, the sodium salt of kappa carragheenan, iota carragheenan, the lithium salt of iota carragheenan, the sodium salt of iota carragheenan, the potassium salt of iota carragheenan, the ammonium salt of iota carragheenan, pectin, sodium pectinate, ammonium pectinate, potassium pectinate, as well as mixtures of such substances, are most preferred. Most preferred mixtures as contemplated herein are those from alginic acid and sodium alginate, more particularly mixtures from alginic acid and sodium alginate in the weight ratio of alginic acid to sodium alginate in the range of from about 1:2 to about 2:1, preferably in the range from about 0.8 to about 1.25, most preferably in the weight ratio of about 1:1.

The aforementioned polysaccharides and/or the mentioned salts demonstrate a thickening behavior that is largely independent of pH. However, they gel in contact with certain polyvalent ions such as calcium, strontium-, barium- and aluminum ions. Moreover, kappa carragheenan also gels in contact with potassium ions.

To the extent required by the present application, this behavior is used to produce a film on the skin, before a reshaping composition or a dye composition is applied, which prevents or at least significantly reduces contact with the skin-irritating hair treatment agent.

For the aforementioned skin protection method as contemplated herein, it is most preferable for the weight ratio of polysaccharide composition (D) to gelling composition (C) to be in the range of from about 0.2:1 to about 2:1, preferably in the range of from about 0.5:1 to about 1.5:1 and is most preferably about 1:1.

### Reshaping Composition (KR)

The reshaping composition (KR) is preferably liquid (from about 0 to about 500 mPas at about 20° C.), medium viscosity (from about >400 to about 4000 mPas at about 20° C.) or creamy (from about >4000 to about 10,000 mPas at about 20° C.).

The reshaping compositions (KR) used as contemplated herein contain at least one keratin-reducing compound, selected from thioglykol acid and the salts thereof and/or thio lactic acid and the salts thereof and/or cysteine and/or acetyl cysteine and/or cysteamine and the salts thereof, as well as mixtures of said keratin-reducing compounds. Preferred reshaping compositions (KR) used as contemplated herein are exemplified in that the at least one keratin-reducing compound is contained in a total quantity of from about 0.1 to about 20 wt. %, preferably from about 1 to about 15 wt. %, most preferably from about 3 to about 13 wt. %, in each case relative to the weight of the agent (KR). Agents (KR) containing thioglykol acid and the salts thereof and/or thio lactic acid and the salts thereof in a total quantity of from about 5 to about 20 wt. %, preferably from about 7 to about 15 wt. %, most preferably from about 10 to about 13 wt. %, in each case relative to the weight of the agent (KR).

### Alkalizing Agent

The reshaping composition (KR) contains at least one alkalizing agent, selected from ammonium hydroxide, monoethanolamine and ammonium carbonate, as well as mixtures thereof.

Preferably, the reshaping composition (KR) contains ammonium hydroxide, i.e. ammonia in the form of its hydrous solution. The corresponding hydrous ammonia solutions can be from about 10 to about 35% solutions (calculated in wt. % of about 100 g hydrous ammonia solution correspondingly containing from about 10 to about 35 g of ammonia). Ammonia in the form of a from about 20 to about 30 wt. % solution is preferably used, most preferably in the form of about 25 wt. % solution.

In a most preferred embodiment, the compositions (KR) used as contemplated herein are exemplified in that they contain ammonium hydroxide in a quantity from about 0.1 to about 5 wt. %, preferably from about 0.2 to about 3 wt. %, more preferably from about 0.3 to about 2 wt. % and most preferably from about 0.4 to about 1.5 wt. %—relative to the weight of the composition (KR) used as contemplated herein.

In addition to and/or instead of ammonium hydroxide, compositions (KR) preferably used as contemplated herein contain monoethanolamine.

In order to achieve a maximum odor minimization and to optimize the reshaping properties, monoethanolamine is used in a total quantity of from about 0.1 to about 6.0 wt. %, preferably from about 0.5 to about 4 wt. %, more preferably from about 1 to about 3.0 wt. % and most preferably from about 2.0 to about 2.5 wt. %—relative to the weight of the composition used as contemplated herein.

In addition to and/or instead of ammonium hydroxide or monoethanolamine, compositions (KR) preferably used as contemplated herein contain ammonium carbonate.

In order to achieve a maximum odor minimization and to optimize the reshaping properties, ammonium carbonate is used in a total quantity of from about 0.1 to about 6.0 wt. %, preferably from about 0.5 to about 4 wt. %, more preferably from about 1 to about 3.0 wt. % and most preferably from about 2.0 to about 2.5 wt. %—relative to the weight of the composition used as contemplated herein.

The reshaped keratin fibers are fixed only through the influence of an oxidant. Hydrogen peroxide is typically used for this purpose. In a preferred embodiment, the hydrogen peroxide is used as a hydrous solution. Oxidant preparations preferred as contemplated herein are exemplified in that they contain from about 1.0 to about 23.0 wt. %, preferably from about 2.5 to about 21.0 wt. %, more preferably from about 4.0 to about 20.0 wt. % and most preferably from about 5.0 to about 18.0 wt. % of hydrogen peroxide (calculated as about 100% H₂O₂).

It has proven advantageous for the oxidant preparations for stabilizing the hydrogen peroxide as contemplated herein contain at least one stabilizer or chelating agent. Most preferred stabilizers complexing agents are, more particularly, EDTA and EDDS, and phosphonates, more particularly 1-hydroxyethane-1,1-diphosphonate (HEDP) and/or ethylenediamine tetramethylene phosphonate (EDTMP) and/or diethylenetriamine pentamethylene phosphonate (DTPMP) or the sodium salts thereof.

In a more preferred embodiment of the present disclosure, the compositions (C) and (D) as contemplated herein are free of anionic or cationic associative polymers, which are substituted with possibly ethoxylated, C₆-C₂₄-alkyl- or C₆-C₂₄-alkenyl groups. In a most preferred embodiment of the present disclosure, the compositions (KR), (C) and (D) used as contemplated herein and the fixing agent are free of anionic or cationic associative polymers, which are substituted with possibly ethoxylated C₆-C₂₄-Alkyl- or C₆-C₂₄-alkenyl groups and selected from acrylates/Ceteth-20 Itaconate copolymers, Polyurethane-39-polymers, acrylates/Beheneth-25 methacrylate copolymers and acrylates/C10-30 alkyl acrylate cross polymers.

### Organic Solvents

Moreover, all compositions used as contemplated herein may contain at least one organic solvent, which is preferably selected from one or more mono- or polyvalent alcohols having from about 2 to about 9 carbon atoms, polyethylenglycols having from about 2 to about 20 ethylenglycol units, as well as mixtures of said solvents, preferably in a total quantity from about 0.001 to about 80 wt. %, more preferably from about 0.01 to about 50 wt. %, even more preferably from about 0.5 to about 20 wt. %, most preferably from about 1.5 to about 10 wt. %, in each case relative to the weight of the composition used as contemplated herein. Preferred organic solvents are selected from C₁-C₄-alcohols, more particularly ethanol and isopropanol, also selected from polyvalent alcohols having from about 2 to about 9, preferably selected from 1,2-propylenglycol, 2-methyl-1,3-propandiol, glycerine, butylenglycols, such as 1,2-butylenglycol, 1,3-butylenglycol and 1,4-butylenglycol, pentylenglycols, such as 1,2-pentandiol and 1,5-pentandiol, hexandiols, such as 1,2-hexandiol and 1,6-hexandiol, hexantriols, such as 1,2,6-hexantriol, 2-ethyl-2-hydroxymethyl-1,3-propandiol, dipropylenglycol, tripropylenglycol, diglycerine and triglycerine, and also selected from polyethylenglycols having from about 2 to about 20 ethylenglycol units, more particularly PEG-3, PEG-4, PEG-6, PEG-7, PEG-8, PEG-9, PEG-10, PEG-12, PEG-14, PEG-16, PEG-18 and PEG-20, as well as mixtures thereof, PEG-3 to PEG-8 being preferred. The use of mixtures of the aforementioned substances is like preferred as contemplated herein.

### Gelling Composition (C)

In order to produce the odor-barrier layer from polysaccharide gel as contemplated herein in situ on the skin, a gelling composition (C) is applied to the skin, for example with a brush or similar applicator, or by spraying, which is most preferred, wherein the gelling composition (C) contains:
c)i. from about 0.1 to about 3 wt. %, preferably from about 0.2 to about 2 wt. %, more preferably from about 0.4 to about 1 wt. %, relative to the weight of the composition (C), at least one salt of a multivalent metal ion, selected from calcium-, strontium-, barium- and aluminum salts having a water solubility at about 20° C. of at least about 500 mg/l, and also if the polysaccharide, which forms a gel with calcium ions, comprises kappa carragheenan or a salt thereof, the gelling composition (C) containing from about 0.1 to about 3 wt. %, preferably from about 0.2 to about 2 wt. %, more preferably from about 0.4 to about 1 wt. %, relative to the weight of the composition (C), at least one salt, selected from potassium, calcium-, strontium-, barium- and aluminums salts having a water solubility at about 20° C. of at least about 500 mg/l,
c)ii. from about 50 to about 99 wt. %, relative to the weight of the composition (C), water,
c)iii. optionally at least one cation polymer, which is preferably selected from cationized guar ethers.
c)iv. wherein the gelling composition (C) has a pH value in the range from about 4.5 to about 7.5, preferably in the range of from about 5 to about 7, most preferably from about 5.5 to about 6.5, measured at about 20° C.;

### Gel-Forming Salt/Water Solubility

At about 20° C., the at least one salt c)i has a water solubility of at least about 500 mg/l, preferably of at least about 40 g/l, more preferably of at least about 200 g/l, and most preferably of at least about 350 g/l. The preferred calcium chloride as contemplated herein has a water solubility of about 740 g/l at about 20° C. Other most preferred salts c)i as contemplated herein are selected from calcium acetate (water solubility about 374 g/l), calcium lactate, calcium gluconate, calcium gluconate lactate. Aluminum chloride, aluminum hydroxychloride, aluminum sulfate, aluminum lactate, aluminum acetate, aluminum gluconate are likewise preferred. Potassium chloride, potassium acetate, potassium lactate, potassium gluconate and potassium sulfate are preferred for the gelling of kappa carragheenan, potassium, chloride, potassium lactate, and potassium gluconate being most preferred. Strontium chloride and barium chloride are also suitable. Preferably, mixtures of the aforementioned salts can also be contained.

The gelling composition (C) contains, in each case relative to its weight, from about 50 to about 99 wt. %, preferably from about 70 to about 95 wt. %, more preferably from about 75 to about 92 wt. % water.

The gelling composition (C) has a pH value in the range from about 4.5 to about 7.5, preferably in the range of from about 5 to about 7, more preferably from about 5.5 to about 6.5, measured at about 20° C. The setting of an acid or an alkali pH depends in part whether the viscosity of the composition (C) for improving the application characteristics thereof, more particularly the spray pattern, is to be increased by employing a thickening agent. Cationic polymers are particularly suitable for promoting moderate thickening of the composition (C). Preferred cationic polymers are selected from cationic guar derivatives, particularly from cationized guar ethers, more particularly from polymers having the INCI designations Hydroxypropyl Guar Hydroxypropyltrimonium Chloride and Guar hydroxypropyltrimonium Chloride. Insofar as the gelling composition (C) contains a cationic polymer, said polymer is contained in a total quantity of from about 0.01 to about 1 wt. %, preferably from about 0.1 to about 0.8 wt. %, more preferably from about 0.2 to about 0.5 wt. %, in each case relative to the weight of the composition (C). Insofar as the gelling composition (C) contains a cationic polymer, said polymer preferably has a pH value in the range of from about 4 to about 6.5, measured at about 20° C.

Just like for the other compositions used as contemplated herein, the pH value is set by employing typical pH setting agents, more particularly citric acid, lactic acid, NaOH, KOH and similar cosmetically-tolerable acids and alkalies.

In a preferred embodiment of the reshaping method as contemplated herein, the gelling composition (C) is produced in-situ or only from about 0.01 to about 24 hours hours before the method as contemplated herein is applied by mixing a solid, preferably powdery gelling composition (C′), containing the at least one salt mentioned under d)i in powder form and optionally at least one cation polymer, which is preferably selected from cationized guar ethers, in powder form, with water d) iii, Most preferably, the gelling composition (C′) additionally contains a solid, preferably powdery, pH setting agent, e.g. an acid such as citric acid, and/or an alkalizing agent, such as sodium silicate.

### Polysaccharide Composition (D)

A particularly preferred embodiment of the skin protection agent as contemplated herein is exemplified in that the at least one polysaccharide, which can form a gel with calcium ions in an aqueous medium and which is preferably selected from alginic acid, kappa carragheenan, iota carragheenan and pectin, and/or at least one salt of the aforementioned polysaccharides, selected from the alkali metal-, ammonium-, mono-, di- and trialkyl ammonium-, mono-, di- and trialkanolammonium- and magnesium salts, wherein in the case of salts of the kappa carragheenan, the alkali metal salt thereof is selected only from lithium-and sodium salts, is prepared in the form of a polysaccharide composition (D), containing
iii.1 at least one polysaccharide, which can form a gel with calcium ions in a hydrous medium and which is preferably selected from alginic acid, kappa carragheenan, iota carragheenan and pectin, and/or at least one salt of the aforementioned polysaccharides, selected from the alkali metal-, ammonium-, mono-, di- and trialkyl ammonium-, mono-, di-and trialkanol ammonium- and magnesium salts, preferably in a total quantity of from about 0.1 to about 5 wt.-%, more preferably from about 0.5 to about 2 wt.-%, most preferably from about 0.5 to about 2.5 wt. %, in each case relative to the weight of the composition (D), wherein the case of salts of the kappa carragheenan, the salt thereof is selected only from lithium- and sodium salts,
iii.2 from about 50 to about 99.9, preferably from about 60 to about 95, most preferably from about 80 to about 90 wt. % water, in each case relative to the weight of the composition (D),
iii.3 optionally at least one alkalizing agent,
iii.4 optionally at least one polymer, containing (meth)acrylic acid, (meth)acrylate-, (meth)acrylamide- and/or 2-acrylamido-2-methylpropansulfonic acid monomers, preferably in a total quantity of from about 0.05 to about 3 wt. %, more preferably from about 0.2 to about 0.5 wt. %, relative to the weight of the composition (D),
iii.5. optionally at least one oil in a total quantity from about 0.1 to about 10 wt. %, preferably from about 0.3 to about 5 wt. %, more preferably from about 1 to about 3 wt. %, in each case relative to the weight of the polysaccharide composition (D), the polysaccharide composition (D) optionally being produced by mixing a solid, preferably powdery polysaccharide composition (D′), containing iii.1 and optionally iii.3 and/or optionally iii.4, with the component iii.2 and optionally with the component iii.5 in situ or from about 0.01 to about 24 hours before the actual, skin-irritating, hair treatment method is applied,

In a preferred embodiment of the skin protection method as contemplated herein, the polysaccharide composition (D) is produced in situ or from about 0.01 to about 24 hours before the reshaping method as contemplated herein is applied, by mixing a solid, preferably a powdery, polysaccharide composition (D′), containing i.1 and optionally iii.3 and/or optionally iii.4, with the component ii.2 and optionally with the component ii.5.

For the in situ production of the polysaccharide composition (D), it has proven useful for at least one polysaccharide, which can form a gel with calcium ions in a hydrous medium, to be dissolved in from about 25° C. to about 40° C. warm water containing at least one oil in a total quantity of from about 0.1 to about 10 wt. %, preferably from about 0.3 to about 5 wt. %, most preferably from about 1 to about 3 wt. %, in each case relative to the weight of the polysaccharide composition (D). The oil ought to be contained in the water before before the polysaccharide is added. This significantly accelerates the dissolution of the polysaccharide. Oils most preferred as contemplated herein are selected from natural and synthetic hydrocarbons, preferably from mineral oils, paraffin oils, C₁₈-C₃₀-isoparaffins, more particularly isoeicosan, polyisobutenes and polydecenes, also selected from C₈-C₁₆-isoparaffins, more particularly from isodecane, isododecane, isotetradecane and isohexadecane, as well as mixtures thereof, as well as 1,3-di-(2-ethylhexyl)-cyclohexane.

Other oils preferred as contemplated herein are selected from the esters of linear or branched saturated or unsaturated fatty alcohols with from about 2 to about 30 carbon atoms having linear or branched saturated or unsaturated fatty alcohols having from about 2 to about 30 carbon atoms which can be hydroxylated. This includes cetyl-2-ethylhexanoate, 2-hexyldecylstearate (e.g. Eutanol® G 16 S), 2-hexyldecyllaurate, isodecylneopentanoate, isononylisononanoate, 2-ethylhexylpalmitate (e.g. Cegesoft® C 24) and 2-ethylhexylstearate (e.g. Cetiol® 868). Preference is also given to isopropylmyristate, isopropylpalmitate, isopropylstearate, isopropylisostearate, isopropyloleate, isooctylstearate, isononylstearate, isocetylstearate, isononylisononanoate, isotridecylisononanoate, cetearylisononanoate, 2-ethylhexyllaurate, 2-ethylhexylisostearate, 2-ethylhexylcocoate, 2-octyldodecylpalmitate, butyloctanoic acid-2-butyloctanoate, diisotridecylacetate, n-butylstearate, n-hexyllaurate, n-decyloleate, oleyloleate, oleylerucate, erucyloleate, erucylerucate, ethylene glycoldioleate and ethylene glycoldipalmitate.

Additional oils preferred as contemplated herein are selected from the benzoic acid esters of linear or branched C8-22-alkanols. Particular preference is given to benzoic acid-C12-C15-alkyl esters, e.g. available as the commercial product Finsolv® TN, benzoic acid isostearyl esters, e.g. available as the commercial product Finsolv® SB, ethylhexylbenzoate, e.g. available as the commercial product Finsolv® EB, and benzoic acid octyldocecyl esters, e.g. available as the commercial product Finsolv® BOD.

Further oils as contemplated herein are selected from fatty alcohols having from about 6 to about 30 carbon atoms, which are unsaturated or branched and saturated or branched and unsaturated. The branched alcohols are often also referred to as Guerbet alcohols, as they are obtained by the Guerbet reaction. Preferred alcohols are 2-hexyldecanole (Eutanol® G 16), 2-octyldodecanole (Eutanol® G), 2-ethylhexyl alcohol and isostearylalcohol.

Further preferred oils are selected from Guerbet alcohols and Guerbet alcohol esters, e.g. from the commercial product Cetiol® PGL (2-hexyldecanole and 2-hexyldecyllaurate).

Additional cosmetic oils preferred as contemplated herein are selected from the triglycerides (=triple esters of glycerin) of linear or branched, saturated or unsaturated, possibly hydroxylated C8-30 fatty acids. Particular preference is given to the use of natural oils, such as amaranthus seed oil, apricot kernel oil, arganil, avocado oil, babassu oil, cotton seed oil, borage oil, camel oil, safflower oil, peanut oil, grenadine core oil, grapefruit seed oil, hemp oil, hazelnut oil, hollowseed oil, currant seed oil, jojoba oil, linseed oil, macadamia nut oil, corn oil, almond oil, marula oil, evening primrose oil, olive oil, palm oil, palm kernel oil, parannut oil, pectic oil, peach kernel oil, rapeseed oil, castor oil, sandalwood oil, sanddornkernel oil, sesame oil, soya oil, sunflower oil, grapeseed oil, walnut oil, wild-type oil, wheat germ oil, and the liquid fractions of coconut oil and the like. However, preference is also given to synthetic triglyceride oils, particularly capric/caprylic triglycerides, i.e. the commercial products Myritol® 318 (BASF) or Miglyol® 812 (Hüls) having unbranched fatty acid esters and glyceryl triisostearin with branched fatty acids.

Additional particularly preferred cosmetic oils as contemplated herein are selected from the dicarboxylic acid esters of linear or branched C2-C10 alkanols, particularly diisopropyl adipate, di-n-butyl adipate, di (2-ethylhexyl)) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Additional preferred cosmetic oils as contemplated herein are selected from the adducts of from about 1 to about 5 propylene oxide units on mono- or polyvalent C8-22 alkanols, such as octanol, decanol, decanediol, lauryl alcohol, myristyl alcohol and stearyl alcohol, e.g. PPG-2 myristyl ether and PPG-3 myristyl ether (Witconol® APM).

Additional preferred cosmetic oils as contemplated herein are selected from the adducts of at least about 6 ethylene oxide and/or propylene oxide units on mono- or polyvalent C3-22 alkanols, such as glycerin, butanol, butanediol, myristyl alcohol and stearyl alcohol, which can be optionally esterified, such as PPG-14 butyl ether (Ucon Fluid® AP), PPG-9 butyl ether (e.g. Breox® B25), PPG-10 butanediol (Macol® 57), PPG-15 stearyl ether (Arlamol® E) and glycereth-7-diisononanoate.

Additional preferred cosmetic oils as contemplated herein are selected from the C8-C22 fatty alcohol esters of monovalent or polyvalent C2-C7 hydroxy carboxylic acids, particularly the esters of glycolic acid, lactic acid, malic acid, tartaric acid, citric acid and salicylic acid. Such esters based on linear C14/15 alkanols, such as C12-C15 alkyl lactate, and on C12/13 alkanols branched in 2-position are commercially available under the trade name Cosmacol® from Nordmann Rassmann GmbH & Col, Hamburg, particularly the commercial products Cosmacol® ESI, Cosmacol® EMI and Cosmacol® ETI.

Additional preferred cosmetic oils as contemplated herein are selected from the symmetrical, asymmetrical or cyclical esters of carboxylic acid with C₃₋₂₂ alkanols, C₃₋₂₂ alkanediols or C₃₋₂₂ alkanetriols, e.g. dicaprylylcarbonate (Cetiol® CC) or the esters according to the teaching of DE 19756454 A1, particularly glycerin carbonate.

Additional cosmetic oils that can be preferred as contemplated herein are selected from the esters of dimeric unsaturated C₁₂-C₂₂ fatty acids (dimeric fatty acids) with monovalent linear, branched or cyclical C₂-C₁₈ alkanols or polyvalent linear and branched C₂-C₆ alkanols.

Additional cosmetic oils that are suitable as contemplated herein are selected from the silicone oils, which include, for example, dialkyl- and akylaryl siloxanes, such as cyclopentadienyl, cyclohexsiloxane, dimethylpolysiloxane and methylphenylpolysiloxane, but also hexamethyldisiloxane, octamethyltrisiloxane and decamethyltetrasiloxane. Preference can be given to volatile silicone oils which can be cyclic, such as octamethylcyclotetrasiloxane, decamethylcyclopentasiloxane and dodecamethylcyclohexsiloxane, and mixtures thereof, as are contained, for example, in the commercial products DC 244, 245, 344 and 345 of Dow Corning. Volatile silicone oils are also suitable, particularly hexamethyldisiloxane (L₂), octamethyltrisiloxane (L₃), Decamethyltetrasiloxan (L₄) and any double and trouble mixtures of L₂, L₃ and/or L₄, preferable such mixtures which are commercially available, for example, in the products DC 2-1184, Dow Corning® 200 (0.65 cSt) and Dow Corning® 200 (1.5 cSt) from Dow Corning. Preferred nonvolatile silicone oils are selected from higher molecular linear dimethylpolysiloxanes, commercially available, for example, under the name Dow Corning® around 200 Fluid with kinematic viscosities (about 25° C.) in a range of from about 5 to about 100 cSt, preferably from about 5 to about 50 cSt or also from about 5 to about 10 cSt, and dimethylpolysiloxane with a kinematic viscosity (about 25° C.) of approximately 350 cSt. According to the present disclosure, the use of mixtures of the aforementioned oils is most preferred.

The dissolution of the polysaccharide can also be promoted by adding a surfactant, for example in a total quantity of from about 0.05 to about 3 wt. %, preferably from about 0.1 to about 1 wt. %, more preferably from about 0.3 to about 0.8 wt. %, relative to the weight of the composition (D). However, a surfactant can cause the polysaccharide composition (D), which ought to be shaken to loosen the polysaccharide, to foam too strongly, making it difficult to apply. The polysaccharide composition (D) therefore preferably contains no surfactant.

Most preferably used polysaccharide compositions (D) and (D′) are exemplified in that the at least one polysaccharide, which can form a gel with calcium ions in hydrous medium, is selected from alginic acid, sodium alginate, ammonium alginate, magnesium alginate, monoethanolammoniumalginate, kappa carragheenan, the lithium salt of kappa carragheenan, the sodium salt of kappa carragheenan, iota carragheenan, the lithium salt of iota carragheenan, the sodium salt of iota carragheenan, the potassium salt of iota carragheenan, the ammonium salt of iota carragheenan, pectin, sodium pectinate, ammonium pectinate, potassium pectinate and magnesium pectinate, as well as mixtures of said substances. Most preferred mixtures as contemplated herein are those from alginic acid and sodium alginate, more particularly mixtures from alginic acid and sodium alginate in the weight ratio of alginic acid to sodium alginate in the range of from about 1:2 to about 2:1, preferably in the range from about 0.8 to about 1.25, most preferably in the weight ratio of about 1:1.

Most preferably, the polysaccharide composition (D) contains a mixture of alginic acid and sodium alginate in a total quantity of from about 0.1 to about 5 wt. %, more preferably from about 0.2 to about 3 wt. %, most preferably from about 0.5 to about 2.5 wt. %, in each case relative to the weight of the composition (D), wherein the a weight ratio of alginic acid to sodium alginate is preferably in the range of from about 1:2 to about 2:1, more preferably in the range from about 0.8 to about 1.25, most preferably in the weight ratio of about 1:1.

According to the present disclosure, the polysaccharide composition (D) has a pH value in the range from about 4.5 to about 7.5, preferably in the range from about 5 to about 7, most preferably from about 5.5 to about 6.5, measured at about 20° C.

According to the present disclosure, it may be preferable for the polysaccharide composition (D) polysaccharide composition (D′) to contain at least one polymer, containing (meth)acrylic acid, (meth)acrylate-, (meth)acrylamide- and/or 2-acrylamido-2-methylpropansulfonic acid monomers, preferably in a total quantity of from about 0.05 to about 3 wt. %, more preferably from about 0.2 to about 0.5 wt. %, relative to the weight of the composition (D), The addition of said polymers serves primarily to improve the solubility of the polysaccharide during the in situ production of the composition (D). Preferably, the at least one polymer, containing (meth)acrylic acid (meth)acrylate-, (meth)acrylamide- and/or 2-acrylamido-2-methylpropansulfonic acid monomers, is selected from cross-linked homopolymers of the acrylic acid, cross-linked homopolymers of 2-acrylamido-2-methylpropansulfonic acid, cross-linked copolymers including of acrylic acid—and 2-acrylamido-2-methylpropansulfonic acid units, cross-linked copolymers including of 2-methyl-2[(1-oxo-2-propenyl)amino]-1-propansulfonic acid units and hydroxyethylacrylate units, cross-linked copolymers including of acrylamide- and 2-acrylamido-2-methylpropansulfonic acid units, cross-linked copolymers including of methacrylic acid units and C1-C4-alkylacrylate units, cross-linked copolymers consiting of acrylic acid units and C1-C4-alkylmethacrylate units, as well as mixtures thereof.

Most preferably, the at least one polymer, containing (meth)acrylic acid-, (meth)acrylate-, (meth)acrylamide- and/or 2-acrylamido-2-methylpropansulfonic acid monomers, having no monomers, which are substituted with possibly ethoxylated, C₆-C₂₄-alkyl-or C₆-C₂₄-alkenyl groups;

A further subject matter of the present disclosure is a kit for hair and skin treatment, comprising:
at least one hair treatment kit compartment containing the hair treatment agent for dyeing and/or for permanently reshaping hair;
at least one hair protection compartment containing the agent for protecting the skin against contact with the hair treatment agent, the skin protection compartment containing the compositions (D) and (C):
   I. A polysaccharide composition (D), containing
d)i. at least one polysaccharide, which can form a gel with calcium ions in an aqueous medium and which is preferably selected from alginic acid, kappa carragheenan, iota carragheenan and pectin, and/or at least one salt of the aforementioned polysaccharides, selected from the alkali metal-, ammonium-, mono-, di- and trialkyl ammonium-, mono-, di-and trialkanolammonium- and magnesium salts, wherein in the case of salts of the kappa carragheenan, the alkali metal salt thereof is selected only from lithium- and sodium salts, mixtures of alginic acids and sodium alginate being most preferred,
d)ii. at least one alkalizing agent, preferably selected from powdery sodium silicates, most particularly powdery sodium meta silicates,
d)iii. optionally, at least one polymer, containing (meth)acrylic acid-, (meth)acrylate-, (meth)acrylamide- and/or 2-acrylamido-2-methylpropansulfonic acid monomers, preferably having no monomers, which are substituted with possibly ethoxylated, C₆-C₂₄-alkyl- or C₆-C₂₄-alkenyl groups;
   II. a gelling composition (C′), containing
c)i. at least one salt of a polyvalent metal ion selected from calcium-, strontium-, barium- and aluminum salts having a water solubility at about 20° C. of at least about 500 mg/l, and also if the polysaccharide, which forms a gel with calcium ions, comprises kappa carragheenan or a salt thereof, at least one salt selected from potassium-, calcium-, strontium-, barium- and aluminum salts having a water solubility at about 20° C. of at least about 500 mg/l;
c)ii. optionally at least one cation polymer, which is preferably selected from cationized guar ethers.

For kits as contemplated herein, the statements made above on the reshaping method as contemplated herein apply mutatis mutandis.

Preferred kits as contemplated herein have the advantage that the essential ingredients of the compositions (C) and (D) can be commercialized in a space- and packaging-saving manner, for example is sachets, which preferably have a vapor-impermeable layer. For example, the hairdresser can produce the compositions (C) and (D) themselves simply by mixing the compositions (C′) and (D′) with water and/or an oily hair care composition. Preferably, the kit also comprises instructions for use for applying the preferred skin protection method as contemplated herein.

A further subject matter of the present disclosure is the use of at least one polysaccharide salt, selected from calcium alginate, potassium kappa carragheenat, calcium kappa carragheenat, calcium iota carragheenat, calcium pectinate, aluminum alginate, aluminum kappa carragheenat, aluminum iota carragheenat, aluminum pectinate, strontium alginate, strontium kappa carragheenat, strontium iota carragheenat, strontium pectinate, barium alginate, barium kappa carragheenat, barium iota carragheenat, barium pectinate, as well as mixtures of said polysaccharide salts, for coating keratinous fibers in a method for the permanent reshaping, more particularly for straightening or perming, keratinous fibers, wherein the reshaping method is preferably a method according to one of claims 1-7 or 9.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1-6. (canceled)

7. A method for protecting the skin against contact with a hair treatment agent during a hair treatment, the method comprising:
(a) applying a polysaccharide composition (D) onto the hair, wherein the polysaccharide composition (D) comprises:
(a1) from about 0.1 to about 5 wt.-%, relative to the total weight of the polysaccharide composition (D), of a polysaccharide adapted to form a gel with calcium ions in a hydrous medium;
(a2) from about 50 to about 99.9 wt.-%, relative to the total weight of the polysaccharide composition (D), of water;
(a3) optionally at least one alkalizing agent;
(a4) optionally at least one polymer, comprising (meth)acrylic acid, (meth)acrylate-, (meth)acrylamide- and/or 2-acrylamido-2-methylpropansulfonic acid monomers; and
(a5) optionally from about 0.1 to about 10 wt. %, relative to the total weight of the polysaccharide composition (D), of at least one oil;
(b) applying a gelling composition (C) onto the hair, wherein the gelling composition comprises:
(b1) from about 0.1 to about 3 wt. %, relative to the total weight of the gelling composition (C), of at least one salt of a polyvalent metal ion, selected from calcium salt, strontium salt, barium salt and aluminum salt having a water solubility at 20° C. of at least about 500 mg/l;
(b2) from about 50 to about 99 wt. %, relative to the total weight of the composition (C), of water;
(b3) optionally at least one cation polymer;
wherein the gelling composition (C) has a pH value of from about 4.5 to about 7.5, measured at 20° C., and
wherein the salts of the gelling composition (C) are not included in the polysaccharide composition (D).

8. The method of claim 7 wherein: (a) applying the polysaccharide composition (D) onto the hair is performed before (b) applying the gelling composition (C) onto the hair.

9. The method of claim 7 wherein: (b) applying the gelling composition (C) onto the hair is performed before (a) applying the polysaccharide composition (D) onto the hair.

10. The method of claim 7 wherein the (a1) at least one polysaccharide is selected from alginic acid; kappa carragheenan; iota carragheenan; pectin; at least one salt of alginic acid, iota carragheenan, and pectin selected from alkali metal salt, ammonium salt, mono-, di- and trialkyl ammonium salt, mono-, di- and trialkanol ammonium salt, and magnesium salt; kappa carragheenan; and/or at least one salt of kappa carragheenan selected from lithium salt and sodium salt.

11. The method of claim 7 wherein the (a1) at least one polysaccharide is selected from alginic acid; kappa carragheenan; iota carragheenan; pectin; at least one salt of alginic acid, iota carragheenan, and pectin selected from alkali metal salt, ammonium salt, mono-, di- and trialkyl ammonium salt, mono-, di- and trialkanol ammonium salt, and magnesium salt; kappa carragheenan; and/or at least one salt of kappa carragheenan selected from lithium salt and sodium salt; and wherein the gelling composition (C) comprises from about 0.1 to about 3 wt. %, relative to the total weight of the composition (C), of at least one salt, selected from potassium salt, calcium salt, strontium salt, barium salt and aluminums salt having a water solubility at 20° C. of at least about 500 mg/l.

12. The method of claim 7 further comprising producing the polysaccharide composition (D) by mixing a solid polysaccharide composition (D′), comprising the a1) polysaccharide and optionally the a3) at least one alkalizing agent and/or optionally a4) the at least one polymer, with the a2) water and optionally with the a5) at least one oil in situ or from about 0.01 to about 24 hours before the hair treatment method is applied.

13. The method of claim 7 wherein the gelling composition (C) is produced in-situ or from about 0.01 to about 24 hours before (b) applying the gelling composition (C) onto the hair, by mixing a solid gelling composition (C′), comprising the (b1) at least one salt of a polyvalent metal ion in powder form and optionally at least one cation polymer, in powder form, with the (b3) water.

14. The method of claim 7 wherein the (a4) at least one polymer, comprising (meth)acrylic acid (meth)acrylate-, (meth)acrylamide- and/or 2-acrylamido-2-methylpropansulfonic acid monomers, is selected from cross-linked homopolymers of the acrylic acid, cross-linked homopolymers of 2-acrylamido-2-methylpropansulfonic acid, cross-linked copolymers comprising acrylic acid- and 2-acrylamido-2-methylpropansulfonic acid units, cross-linked copolymers comprising 2-methyl-2[(1-oxo-2-propenyl)amino]-1-propansulfonic acid units and hydroxyethylacrylate units, cross-linked copolymers comprising acrylamide- and 2-acrylamido-2-methylpropansulfonic acid units, cross-linked copolymers comprising methacrylic acid units and C1-C4-alkylacrylate units, cross-linked copolymers comprising acrylic acid units and C1-C4-alkylmethacrylate units, and/or mixtures thereof.

15. The method of claim 7 wherein the weight ratio of polysaccharide composition (D) to gelling composition (C) is from about 0.2:1 to about 2:1.

16. The method of claim 7 wherein the weight ratio of polysaccharide composition (D) to gelling composition (C) is from about 0.5:1 to about 1.5:1.

17. The method of claim 7 wherein the weight ratio of polysaccharide composition (D) to gelling composition (C) is about 1:1.

18. A kit for hair and skin treatment comprising:
at least one hair treatment kit compartment comprising a hair treatment agent for dyeing and/or for permanently reshaping hair;
at least one skin protection compartment comprising an agent for protecting the skin against contact with the hair treatment agent, the skin protection compartment comprising a gelling composition (C) and polysaccharide composition (D), wherein:
the polysaccharide composition (D) comprises:
(a1) at least one polysaccharide adapted to form a gel with calcium ions in a hydrous medium;
(a3) at least one alkalizing agent;
(a4) optionally at least one polymer, comprising (meth)acrylic acid, (meth)acrylate-, (meth)acrylamide- and/or 2-acrylamido-2-methylpropansulfonic acid monomers which are substituted with C₆-C₂₄-alkyl- or C₆-C₂₄-alkenyl groups or ethoxylated C₆-C₂₄-alkyl- or C₆-C₂₄-alkenyl groups;
the gelling composition (C) comprises:
(b1) at least one salt of a polyvalent metal ion, selected from calcium salt, strontium salt, barium salt and aluminum salt having a water solubility at 20° C. of at least about 500 mg/l; and
(b3) optionally at least one cation polymer.

19. A method for protecting skin, the method comprising applying to the skin at least one polysaccharide salt selected from calcium alginate, potassium kappa carragheenat, calcium kappa carragheenat, calcium iota carragheenat, calcium pectinate, aluminum alginate, aluminum kappa carragheenat, aluminum iota carragheenat, aluminum pectinate, strontium alginate, strontium kappa carragheenat, strontium iota carragheenat, strontium pectinate, barium alginate, barium kappa carragheenat, barium iota carragheenat, barium pectinate, as well as mixtures of said polysaccharide salts.

20. The method of claim 19 wherein the method protects skin against contact with a hair treatment agent during a hair treatment.

21. The method of claim 20 wherein the hair treatment is a permanent reshaping treatment.

22. The method of claim 20 wherein the hair treatment is a keratinous fiber dyeing treatment.
